(19) United States
(12) Patent Application Publication
(10) Pub. No.: US 2012/0202754 A1
(43) Pub. Date: Aug. 9, 2012

(54) ENHANCED SUBSTRATES FOR THE PROTEASE ACTIVITY OF SEROTYPE A BOTULINUM NEUROTOXIN

(76) Inventors: James J. Schmidt, Mount Airy, MD (US); Robert G. Stafford, Ranson, WV (US)

(21) Appl. No.: 13/502,504

(22) PCT Filed: Oct. 15, 2010
(86) PCT No.: PCT/US10/52812

§ 371 Date: Apr. 17, 2012

(30) Foreign Application Priority Data
Oct. 18, 2009 (US) 61252675

(51) Int. Cl. A61K 38/10; A61P 43/00; C12Q 1/37; C07K 7/08; G01N 21/64

(52) U.S. Cl. 514/21.5; 530/327; 436/501; 435/7.1; 435/24

(57) ABSTRACT

Substrates of botulinum toxin serotype A (BoNT A), kits comprising the substrates, and methods of using the substrates are disclosed. BoNT A cleaves SNAP-25 and the substrates that are based upon a portion of SNAP-25. Various amino acid modifications to the sequence of the peptide based on SNAP-25 are performed. Fluorescence labels that act as donors and acceptors may be added to the substrate to aid in the study of BoNT A.

### CROSS-REFERENCE TO RELATED APPLICATIONS

The present application claims priority to U.S. Provisional Patent Application No. 61/252,675, filed Oct. 18, 2009, the entire disclosure of which is herein incorporated by reference.

### STATEMENT REGARDING FEDERALLY SPONSORED RESEARCH OR DEVELOPMENT

This work described in this disclosure was made with government support under the Defense Threat Reduction Agency Research Plan 3.10023_{—}07_RD_B of the Department of Defense. The United States Government has certain rights in the invention.

### FIELD

The field of the disclosure is Botulinum neurotoxins and substrates thereof.

### BACKGROUND

Botulinum neurotoxins (BoNTs) are proteins produced by various strains of Clostridium botulinum, Clostridium butyricum, and Clostridium baratii, and are possibly the most toxic substances known (1-3). There are seven BoNT serotypes, designated A through G, each expressed as a single-chain protein of 150 kDa. Clostridium botulinum produces all seven serotypes while the other two strains produce one serotype each. The seven serotypes of botulinum neurotoxin (BoNTs) are zinc metalloproteases that cleave and inactivate proteins critical for neurotransmission. Synaptosomal protein of 25 kDa (SNAP-25) is cleaved by BoNTs A, C, and E, while vesicle-associated membrane protein (VAMP) is the substrate for BoNTs B, D, F, and G. Serotype C also cleaves syntaxin. BoNTs are not only medically useful drugs, but are also potential bioterrorist and biowarfare threat agents.

In most serotypes the single-chain protein is cleaved by endogenous bacterial protease(s) to yield the dichain molecule, consisting of a heavy chain (100 kDa) and a light chain (50 kDa), covalently linked by a disulfide bond. Cleavage of a single-chain to a dichain form increases toxicity (30). Both chains must be present in the disulfide-linked holotoxin form to cause botulism; i.e., individual chains are not toxic (2-5). BoNTs block the release of acetylcholine from peripheral cholinergic nerve endings. The heavy chain of BoNT binds to receptors on peripheral cholinergic neurons, leading to internalization of the toxin. The light chain is a zinc metalloprotease. The receptor for BoNT A is the SV2 protein (32). The disulfide between the heavy and light chains of BoNT A is reduced when BoNT A enters the cytosol. Upon escape from endosomes into the neuronal cytosol, the light chain of each BoNT serotype cleaves only one peptide bond in its respective substrate. The synaptosomal protein of 25 kDa (SNAP-25) is the substrate for BoNTs A, C, and E. Serotype C also cleaves syntaxin. Vesicle-associated membrane protein (VAMP) is the target of BoNTs B, D, F, and G. Proteolysis at any one of these sites inactivates neurotransmitter exocytosis. Neurotransmitters such as acetylcholine transmit nerve impulses to signal muscles to contract. Therefore, prevention of neurotransmission leads to the flaccid paralysis of botulism (2, 3). The main forms of botulism are food borne, intestinal, and wound related. BoNT can enter the body by, including but not limited to, inhalation, colonization of the digestive tract, ingesting the toxin from foods, and contamination of a wound. Exposure can occur by, including but not limited to, contaminated food, biowarfare, and cosmetic use of inappropriate amounts of botulinum toxin. The toxin actively passes through the lining of the gut to reach the general circulation.

In order for vesicle fusion to occur with the neuronal membrane, synaptobrevin, present on the synaptic vesicle, must interact with syntaxin and SNAP-25 on the neuronal membrane. The heavy chain of BoNT A is responsible for cell surface binding and membrane translocation. The light chain dissociates from the heavy chain inside the endosome. The light chains of various serotypes of BoNTs cleave the peptide bonds at locations within synaptobrevin, syntaxin, and SNAP-25, therefore preventing fusion between the vesicle and the neuronal membrane and preventing nerve impulse transmission. (31)

Despite the extreme toxicity of BoNT, clinicians use BoNT to treat an ever-expanding variety of human diseases where pathological conditions are caused by unregulated exocytosis of acetylcholine (6-8). BoNT is used to treat blepharospasm, strabismus, muscle spasms, migraines, upper motor neuron syndrome, sweating, cervical dystonia, cerebral palsy, urinary incontinence, and for cosmetic uses. In addition, recent results suggest that BoNT might be useful in cancer therapy, where its effects on tumor vascular structure enhanced the efficacy of radiation treatments and chemotherapy (9). Finally, BoNT heavy chain and genetically inactivated holotoxin have been studied as potential intracellular drug carriers targeted specifically to neurons (10-13). Holotoxin refers to the fully active form of the toxin, containing both subunits.

Unfortunately, BoNT is also a serious biowarfare and bioterrorism threat (1, 14). At present, the only treatment available for patients suffering from systemic botulism is supportive care including administration of specific antibodies to eliminate toxin still in the circulation (antitoxin), and mechanical ventilation, if needed. Recovery can take weeks or months. Because of the large number of serotypes and its use in human medicine, large-scale vaccination of the general population against BoNT is impractical and undesirable. Once inside the neuronal cell, BoNT cannot be neutralized by antibodies. Currently, there are no drugs available that can reach intracellular toxin to reverse or mitigate its effects (1, 7, 15). Because the protease activity of BoNT is required for toxicity, efforts are underway to develop specific inhibitors that might be useful as anti-botulinum drugs, or serve as model compounds for effective drug development (16-23).

FRET substrates for botulinum protease activity have previously been reported (U.S. Pat. Nos. 6,504,006; 6,762,280; 7,034,107; and 7,157,553). Progress in the identification of potential anti-botulinum drugs would be expedited by the availability of substrates suitable not only for high-throughput assays but also for kinetic and mechanistic studies. The present disclosure reports such substrates for BoNT A protease activity, and demonstrates their superiority compared to other substrates.

### BRIEF SUMMARY

An embodiment is an isolated peptide comprising the amino acid sequence X1 X2 R I D X3 A N Q R A T X4 X5 (SEQ ID NO: 1) wherein each of X1 through X5 is chosen from the following: X1 is K, R, ornithine, or 2, 4-diaminobutanoic acid (dbu); X2 is V, 2-aminobutanoic acid (B), I, L, or T; X3 is Q, A, norvaline (nV), B, or E; X4 is K, R, or dbu; and X5 is M or norleucine (nL); except wherein X1=K, X2=T, X3=E, X4=K, and X5=M; X1=K, X2=T, X3=Q, X4=K, and X5=M; or X1=R, X2=T, X3=Q, X4=R, and X5=M. In an embodiment, SEQ ID NO: 1 is modified with a fluorophore located on one side of a BoNT A cleavage site and a quencher located on the other side of the BoNT A cleavage site.

In another embodiment, the isolated peptide comprises the amino acid sequence R V R I D A A N Q R A T R M (SEQ ID NO: 2). In yet another embodiment, the isolated peptide is modified with a fluorophore located on one side of a BoNT A cleavage site and a quencher located on the other side of the BoNT A cleavage site. In an embodiment, the fluorophore is located at one terminus and a quencher is located at the other terminus. In an embodiment, DabcylK is located at the N-terminus and S-fluoresceinyl cysteine is located at the C-terminus.

In another embodiment, the isolated peptide comprises the amino acid sequence R V R I D A A N Q R A T R nL (SEQ ID NO: 3). In an embodiment, the isolated peptide is modified with a fluorophore located on one side of a BoNT A cleavage site and a quencher located on the other side of the BoNT A cleavage site. In another embodiment, the fluorophore is located at one terminus and a quencher is located at the other terminus. In an embodiment, DabcylK is located at the N-terminus and S-fluoresceinyl cysteine is located at the C-terminus.

An embodiment is a kit to detect the presence of BoNT A in a sample comprising the isolated peptide SEQ ID NO: 1 modified with a fluorophore located on one side of a BoNT A cleavage site and a quencher located on the other side of the BoNT A cleavage site; polymeric beads coated with antibodies specific for BoNT A; and polymeric beads coated with immunoglobulins not specific for BoNT A. In an embodiment, the kit further comprises lyophilized BoNT A Lc. In another embodiment, the kit further comprises a pH-buffering compound in a first container. In yet another embodiment, the pH-buffering compound is selected from the group consisting of sodium hydroxyethylpiperazine sulfonate (HEPES) and sodium phosphate. In still another embodiment, the pH-buffering compound is in dry form. In an embodiment, the kit further comprises bovine serum albumin. In another embodiment, the kit further comprises polysorbate 20. In an embodiment, the polysorbate 20 is added to a final concentration of 0.05-0.10% (v/v). In an embodiment, the kit further comprises a reducing agent in a first container. In an embodiment, the reducing agent is selected from the group consisting of dithiothreitol and tris-(carboxyethyl)-phosphine. In another embodiment, the kit further comprises a zinc salt in a first container. In an embodiment, the zinc salt is selected from the group consisting of zinc chloride and zinc acetate. In yet another embodiment, the kit further comprises purified water in a second container. In still another embodiment, the kit further comprises purified dimethylsulfoxide in a third container. In an embodiment, the lyophilized BoNT A Lc comprises a stabilizing excipient. In another embodiment, the isolated peptide is in dry form.

An embodiment is a method of detecting the presence of BoNT A in a sample comprising placing the sample in solution in a pH-buffering compound; mixing the sample in the pH-buffering compound with polymeric beads coated with antibodies specific for BoNT A to provide a test assay; mixing the sample in the pH-buffering compound with polymeric beads with immunoglobulins not specific for BoNT A to provide a control assay; incubating the sample with the pH-buffering compound with polymeric beads coated with antibodies specific for BoNT A to provide a test assay; incubating the sample with the pH-buffering compound with polymeric beads with immunoglobulins not specific for BoNT A to provide a control assay; washing the polymeric beads coated with antibodies specific for BoNT A with the pH-buffering compound; washing the polymeric beads without antibodies with the pH-buffering compound; suspending the beads from the test assay in a first container comprising the pH-buffering compound, dithiothreitol, and a zinc salt; suspending the beads from the control assay a second container comprising the pH-buffering compound, dithiothreitol, and a zinc salt; adding isolated peptide SEQ ID NO: 1 that is modified with a fluorophore located on one side of a BoNT A cleavage site and a quencher located on the other side of the BoNT A cleavage site to the test assay and control assay; incubating the isolated peptide with the beads from the test assay; incubating the isolated peptide with the beads from the control assay; separating the beads from the test assay from a test assay solution comprising the isolated peptide; separating the beads from the control assay from a control assay solution comprising the isolated peptide; measuring fluorescence intensity of the test assay solution; measuring fluorescence intensity of the control assay solution; comparing the fluorescence of the test assay solution and the control assay solution; and determining whether BoNT A is present in the sample by whether the fluorescence of the test assay solution is higher than the fluorescence of the control assay solution. In an embodiment, the method further comprises adding bovine serum albumin to the pH buffering compound. In another embodiment, the method further comprises adding polysorbate 20 present to the pH buffering compound. In an embodiment, the polysorbate 20 is added to a final concentration of 0.05-0.10%.

An embodiment is a method of determining the concentration of BoNT A in a test sample comprising placing the sample in solution in a pH-buffering compound; mixing the sample in the pH-buffering compound with polymeric beads coated with antibodies specific for BoNT A to provide a test assay; mixing the sample in the pH-buffering compound with polymeric beads with immunoglobulins not specific for BoNT A to provide a control assay; incubating the sample with the pH-buffering compound with polymeric beads coated with antibodies specific for BoNT A to provide a test assay; incubating the sample with the pH-buffering compound with polymeric beads with immunoglobulins not specific for BoNT A to provide a control assay; washing the polymeric beads coated with antibodies specific for BoNT A with the pH-buffering compound; washing the polymeric beads without antibodies with the pH-buffering compound; suspending the beads from the test assay in a first container comprising the pH-buffering compound, dithiothreitol, and a zinc salt; suspending the beads from the control assay a second container comprising the pH-buffering compound, dithiothreitol, and a zinc salt; adding the isolated peptide SEQ ID NO: 1 that is modified with a fluorophore located on one side of a BoNT A cleavage site and a quencher located on the other side of the BoNT A cleavage to the test assay and control assay; incubating the isolated peptide with the beads from the test assay; incubating the isolated peptide with the beads from the control assay; separating the beads from the test assay from a test assay solution comprising the isolated peptide; separating the beads from the control assay from a control assay solution comprising the isolated peptide; measuring fluorescence intensity of the test assay solution; measuring fluorescence intensity of the control assay solution; comparing the fluorescence of the test assay solution and the control assay solution; and determining the concentration of BoNT A in the sample by comparison of fluorescence intensity of the test assay solution with a standard curve prepared using known concentrations of BoNT A Lc. In an embodiment, the method further comprises adding bovine serum albumin to the pH buffering compound. In another embodiment, the method further comprises adding polysorbate 20 present to the pH buffering compound. In an embodiment, the polysorbate 20 is added to a final concentration of 0.05-0.10%.

An embodiment is a method for measuring the activity of BoNT A comprising incubating SEQ ID NO: 1 with BoNT A to form a sample; injecting the sample onto an HPLC column; preparing a chromatogram of the elution of various components of the sample; analyzing the chromatogram to determine how much of the isolated peptide was cleaved based upon the size of peaks correlating to the isolated peptide and cleaved portions of the isolated peptide.

An embodiment is a method for identifying a BoNT A inhibitor comprising incubating BoNT A with a potential inhibitor to form a first sample; incubating BoNT A without a potential inhibitor to form a second sample; adding SEQ ID NO: 1 to the first sample; adding SEQ ID NO: 1 to the second sample; stopping the reactions by adding acid to the first and second sample; injecting the first sample onto an HPLC column; preparing a chromatogram of the elution of various components of the first sample; injecting the second sample onto an HPLC column; preparing a chromatogram of the elution of various components of the second sample; analyzing the chromatogram for the first sample and the second sample to determine how much of the isolated peptide was cleaved based upon the size of peaks correlating to the isolated peptide and cleaved portions of the isolated peptide; and determining that a potential inhibitor of BoNT A is a BoNT A inhibitor if the size of the peak correlating to the isolated peptide for the first sample is taller than the size of the peak for the isolated peptide the second sample.

An embodiment is a method of treating an individual in need of treatment for a disorder due to BoNT A comprising administering a composition comprising SEQ ID NO: 1.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 depicts an HPLC chromatogram illustrating hydrolysis of P6 (SEQ ID NO: 2) catalyzed by BoNT A light chain.

FIG. 2 depicts a plot of fluorescence values vs. time in the absence (Blank) and presence (A-Lc) of BoNT A light chain. The substrate is flP6 ([DabcylK] (SEQ ID NO: 2) [SFC]).

FIG. 3A depicts the effects of BoNT A light chain concentrations on hydrolysis of flP6 ([DabcylK] (SEQ ID NO: 2) [SFC]).

FIG. 3B depicts the effects of substrate concentrations on hydrolysis of flP6 ([DabcylK] (SEQ ID NO: 2) [SFC]), catalyzed by BoNT A light chain.

FIG. 4 depicts initial hydrolysis rates (v) of flP6 ([DabcylK] (SEQ ID NO: 2) [SFC]) at various concentrations, catalyzed by BoNT A light chain.

### DETAILED DESCRIPTION

The disclosure relates to botulinum toxin and substrates thereof. It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, methods, procedures and components have not been described in detail so as not to obscure the embodiments described herein.

The following definitions and explanations are meant and intended to be controlling in any future construction unless clearly and unambiguously modified in the following Description or when application of the meaning renders any construction meaningless or essentially meaningless. In cases where the construction of the term would render it meaningless or essentially meaningless, the definition should be taken from Webster's Dictionary, 3rd Edition. Definitions and/or interpretations should not be incorporated from other patent applications, patents, or publications, related or not, unless specifically stated in this specification or if the incorporation is necessary for maintaining validity.

The term “FRET substrate”, as used herein, refers to a peptide substrate with a fluorophore and quencher placed on opposite sides of the BoNT A cleavage site within the substrate.

The term “HPLC”, as used herein, refers to high performance liquid chromatography. The term HPLC includes RPHPLC (reverse phase high performance liquid chromatography). RPHPLC is a chromatographic method that uses a non-polar stationary phase.

The term “2,4-diaminobutanoic acid”, as used herein, is also referred to as 2,4-diaminobutyric acid and abbreviated as dab or dbu.

The term “2-aminobutanoic acid”, as used herein, is also referred to as 2-aminobutyric acid and abbreviated as B or abu.

The term “polysorbate-20”, as used herein, is also referred to as poly(oxyethylene)ₓ-sorbitane-monolaurate or Tween® 20.

### BoNT A Substrates

Because BoNT protease activity is required for toxicity, inhibitors of that activity might be effective for anti-botulinum therapy. Peptides are described for use in assaying, detecting and/or quantifying the protease activity of serotype A botulinum neurotoxin (BoNT A).

Chromatography-based and fluorigenic substrates for the protease activity of BoNT A having significantly enhanced kinetic properties with regard to rates of hydrolysis and binding affinities, compared to commonly-used BoNT A substrates such as the neuronal protein SNAP-25 and fragments and derivatives thereof have been developed. These substrates may expedite inhibitor discovery. The enhancements include non-conservative structural modifications (four amino acid replacements and three truncations) in a BoNT A substrate peptide which in its original form, corresponded to residues 187 to 203 of SNAP-25. BoNT A cleaves the peptide corresponding to residues 187 to 203 of SNAP-25 between the glutamine at 197 (Q197) and the arginine at 198 (R198). In one embodiment, the hydrophilic residue threonine at 190 (T190) was replaced with a hydrophobic residue valine (V190). In this embodiment, a hydrophilic residue was replaced with a hydrophobic residue. In an embodiment, a negatively-charged residue, glutamic acid at 194 (E194) can be replaced with the neutral, moderately hydrophobic residue, alanine (A194). The minimum substrate required for efficient proteolysis by BoNT A is defined herein. The present disclosure provides new insight with regard to BoNT A substrate recognition mechanism.

All peptides have carboxamide at the C-terminus and N-acetyl at the N-terminus. The standard single-letter abbreviations for naturally-occurring amino acids are employed. Non-standard abbreviations are defined in the list below. All amino acids are of the [L] configuration. Each is a substrate for the protease activity of BoNT A. The general structure is:

(SEQ ID NO: 1)
X1 X2 R I D X3 A N Q R A T X4 X5

X1 through X5 are chosen from the following list:
X1 is K, R, ornithine, or 2,4-diaminobutanoic acid (dbu).
X2 is V, 2-aminobutanoic acid (B), I, L, or T.
X3 is Q, A, norvaline (nV), B, or E.
X4 is K, R, or dbu.
X5 is M or norleucine (nL).

Peptides with any combination of X1 through X5 are disclosed and can be employed as substrates in BoNT A assays based on the post-reaction separation and quantification of hydrolysis products and remaining substrate by several commonly-used procedures, such as reverse-phase high pressure liquid chromatography (RPHPLC), mass spectrometry, or liquid chromatography/mass spectrometry.

The following is an example of a peptide substrate for the protease activity of BoNT A. X1 is R, X2 is V, X3 is A, X4 is R, and X5 is M. This peptide is referred to herein as P6:

(SEQ ID NO: 2)
R V R I D A A N Q R A T R M

Another example of a peptide substrate for the protease activity of BoNT A is the following peptide where X1 is R, X2 is V, X3 is A, X4 is R, and X5 is nL (norleucine):

(SEQ ID NO: 3)
R V R I D A A N Q R A T R nL

### High Performance Liquid Chromatography (HPLC)

In HPLC, a sample is injected onto a column comprising a stationary phase (column material). The mobile phase (buffer or solvent) is moved through the column using a pump. The pump provides the high pressure to move the solvent through the column. A detector provides a retention time for the analyte by measuring the level of a particular characteristic of the solvent, such as absorbance, as it elutes from the column. The analyte is retained in the column longer by interactions with the stationary phase and the analyte progresses through the column. Separation of various components present in the sample is possible as the various components will interact with the stationary phase differently. Separation of the peaks indicating retention time of the various components may be improved by adjusting the HPLC protocol.

### Enzyme Kinetics

The rates at which the enzyme, BoNT A, works were measured for various substrates. Km is the Michaelis constant and it is a measure of the substrate concentration required for effective catalysis to occur. Km is often used to describe the affinity of an enzyme for a substrate. Generally, the lower the Km, the higher the affinity. Kcat provides a measure of the production of product with saturated enzyme. Kcat measure the number of substrate molecules turned over per enzyme molecule per second. Kcat is often referred to as the turnover number. Kcat/Km is a measure of enzyme efficiency.

Förster (Fluorescence) Resonance Energy Transfer

FRET requires the spectral overlap of the donor (fluorophore) emission spectrum and the acceptor (quencher) absorption spectrum. The donor (fluorophore), when excited by energy at the donor's excitation wavelength, emits energy at the donor's emission wavelength. If an acceptor (quencher) is present, in close enough proximity to the donor, and has an absorption wavelength similar to the emission wavelength of the donor, the fluorescence of the donor will be decreased or quenched. When the acceptor is no longer in proximity of the donor, the donor's emission will no longer be quenched and an increase in fluorescence at the donor's emission wavelength will occur. The emission wavelength of the donor is monitored to determine if BoNT A is cleaving a peptide substrate modified with a fluorophore and quencher.

FRET substrates for BoNT A protease activity may contain the substrate sequences disclosed herein. In a FRET protease substrate, a fluorophore and a quencher are added to a substrate on opposite sides of the cleavage site to yield a fluorescence resonance energy transfer (FRET) substrate. The fluorophore and quencher may be placed on opposite ends of the substrate, with the fluorophore on the N-terminus and the quencher on the C-terminus, or vice-versa. Alternatively, the fluorophore and quencher may be placed within the sequence of the substrate, as long as they are on opposite sides of the cleavage site. All such possibilities are understood to be included here. Any fluorophore/quencher pair may be used. Fluorophore: quencher pairs that may be used include, but are not limited to, dinitrophenyl (dnp): N-(7-dimethylamino-4- methylcoumarin-3-yl)iodoacetamide (DACIA), fluorescein-4-(dimethylaminoazo)benzene-4-carboxylic acid (Dabcyl), blue fluorescent protein (BFP): yellow fluorescent protein (YFP), cyan fluorescent protein (CFP):YFP, green fluorescent protein (GFP): red fluorescent protein from Discosoma coral (DsRed), GFP: cyanine3 (Cy3), GFP: mOrange, YFP: red fluorescent protein (RFP), Cy3: cyanine5 (Cy5), and 5-({2-[(iodoacetyl)amino]ethyl}amino)naphthalene-1-sulfonic acid (IAEDANS): 5-iodoacetamidofluorescein (IAF). Initially, fluorescence is relatively low due to the proximity of the quencher. When the substrate is cleaved, the fluorophore and quencher diffuse away from one another and fluorescence increases. The substrate structures disclosed herein include performance-enhancing amino acid sequence modifications.

In various embodiments, substrates disclosed in this disclosure may be used to:
(1) Detect and quantify the presence of BoNT A in clinical, forensic, and other types of samples.
(2) Measure the activity and concentration of BoNT A.
(³) Standardize preparations of BoNT A for laboratory use and in clinical applications.
(4) Search for inhibitors of BoNT A protease activity, to serve as model compounds for anti-botulinum drug development.
(⁵) Investigate the catalytic and substrate recognition requirements of BoNT A.

The substrates exhibit significantly enhanced kinetic properties with regard to rates of hydrolysis and binding affinities, compared to commonly-used BoNT A substrates such as the neuronal protein SNAP-25 and fragments and derivatives thereof. The enhancements include non-conservative structural modifications (amino acid replacements) and truncations in a BoNT A substrate peptide which in its original form, corresponded to residues 189 to 203 of SNAP-25.

Commonly-used substrates for the protease activity of BoNT A are difficult to synthesize and/or purify, offer relatively low sensitivity, in some cases are of low solubility in aqueous buffers, and exhibit kinetic properties that render them unsuitable for use in enzyme mechanistic studies. The substrates herein are easily synthesized and purified, afford relatively high sensitivity, and have kinetic properties appropriate for mechanistic studies and BoNT A inhibitor discovery.

### EXAMPLES

The following examples are included to demonstrate preferred embodiments of the present disclosure. It should be appreciated by those of skill in the art that the techniques disclosed in the examples which follow represent techniques discovered by the inventors to function well and thus can be considered to constitute preferred modes for its practice.

However, those of skill in the art should, in light of the present disclosure, appreciate that many changes can be made in the specific embodiments which are disclosed and still obtain a like or similar result without departing from the spirit or scope of the disclosure. The following Examples are offered by way of illustration and not by way of limitation.

### Example 1

Peptides were synthesized on a Model 431A peptide synthesizer from Applied Biosystems (Foster City, Calif.). FMOC-DABCYL-lysine was purchased from AnaSpec, San Jose, Calif. Iodoacetamidofluorescein was purchased from Thermo Scientific (Pierce), Rockford, Ill. All other chemicals and protected amino acids for peptide synthesis were obtained from Applied Biosystems. All peptides were C-terminal amides, and N-terminal amino groups were acetylated. Peptides were purified by HPLC with gradients of dilute trifluoroacetic acid and acetonitrile using equipment from Waters Associates (Milford, Mass.). Molecular masses of peptides were confirmed by mass spectrometry.

A Rink amide resin may be used for peptide synthesis. Peptide chains are built on the resin and occur in a C-terminal to N-terminal manner. The N-terminus an amino acid monomer is protected by a group such as Fmoc (9-fluorenylmethyloxycarbonyl) or Boc (tert-butoxycarbonyl). The monomer is added to the deprotected N-terminal amine of an amino acid at the end of the chain opposite the resin. The Fmoc or Boc is removed, thus deprotecting the new amino acid at the end of the chain. In an embodiment, a base such as piperidine is used to remove Fmoc. In an embodiment, an acid, such as trifluoroacetic acid (TFA) is used to remove Boc. Repeated cycles of coupling, wash, deprotection, and wash occur. Final cleavage of the peptide chain from the resin is accomplished using TFA. Peptide substrates may be synthesized in any manner known in the art.

### Example 2

BoNT A protease activity was assayed. Recombinant BoNT A light chain (BoNT A Lc) was produced and purified as described (4). Assays were stopped by acidification with trifluoroacetic acid and analyzed by HPLC (21, 24).

Table 1 summarizes the effects of several amino acid substitutions and truncations on BoNT A-catalyzed substrate hydrolysis rates. Peptide P1 (SEQ ID NO: 4) contains residues 187-203 of SNAP-25 and was originally developed as a convenient 17-mer substrate to study the enzymatic activity of BoNT A (24). Replacing T190 with valine to yield P2 (SEQ ID NO: 5) increased the hydrolysis rate by 1.7-fold, while an additional replacement, E194Q ((P3) (SEQ ID NO: 6)), further enhanced the rate.

TABLE 1
Relative hydrolysis rates of BoNT A peptide
substrates¹.

| Peptide | Sequence | Relative v² | |
|---|---|---|---|
| | 187 190 195 200 203 | | |
| P1 | S N K T R I D E A N Q R A T K M L³ | 1.0 | (SEQ ID NO: 4) |
| P2 | S N K V R I D E A N Q R A T K M L | 1.7 ± 0.013 | (SEQ ID NO: 5) |
| P3 | S N K V R I D Q A N Q R A T K M L | 2.2 ± 0.014 | (SEQ ID NO: 6) |
| P4 | S N R V R I D Q A N Q R A T R M L | 2.2 ± 0.027 | (SEQ ID NO: 7) |
| P5 | N R V R I D A A N Q R A T R M | 3.0 ± 0.021 | (SEQ ID NO: 8) |
| P6 | R V R I D A A N Q R A T R M | 3.0 ± 0.0091 | (SEQ ID NO: 2) |
| P7 | V R I D A A N Q R A T R M | 2.1 ± 0.010 | (SEQ ID NO: 9) |

¹Assays contained 0.40 mM substrates.
²Hydrolysis rate in μmoles/min/mg BoNT A Lc
³SNAP-25 residues 187-203

Other substitutions were tested for T190 and E194. For T190, 2-aminobutanoic acid, leucine, and isoleucine were as effective as valine, but alanine caused a decrease in hydrolysis rate. A branched or straight-chain hydrophobic residue is preferred at position 190. For E194, 2-aminobutanoic acid, norvaline, or alanine had the same effect as glutamine, but valine decreased the hydrolysis rate. The key factor was elimination of the negative charge and glutamine or an uncharged non-branched amino acid gave the best results at position 194.

Replacing both lysines (K189 and K201) with arginine ((P4) (SEQ ID NO: 7)) had no effect. The effects of replacing the lysines with ornithine, 2, 4-diaminobutanoic acid, 2,3-diaminopropanoic acid, and norleucine were determined. With norleucine in place of one or both lysines, only trace hydrolysis was detected, demonstrating that BoNT A prefers basic residues at both locations. For the other substitutions, the order of effectiveness with regard to hydrolysis rate was R=K>ornithine>2,4-diaminobutanoic acid>>2,3-diaminopropanoic acid. Therefore, a basic residue with a 6-carbon side chain was best at positions 189 and 201. In many of the peptides tested, arginine is used at both locations to enhance solubility in aqueous solutions.

Previous results showed that elimination of the C-terminal leucine from the native-sequence 17-mer had no effect on hydrolysis rate, but further truncation (i.e. eliminating methionine) drastically reduced the rate (24). These observations held true for the substituted peptides in Table 1. In contrast, elimination of the N-terminal serine (S187) actually enhanced cleavage velocity (compare P4 (SEQ ID NO: 7) and P5 (SEQ ID NO: 8), Table 1). S187 in SNAP-25 does not interact with BoNT A light chain (28). Therefore, its absence is expected to reduce the entropy of unbound P5 (SEQ ID NO: 8), compared to P4 (SEQ ID NO: 7), lowering the binding constant. Additional truncation of N188 ((P6) (SEQ ID NO: 2)) had no effect, but removal of R189 ((P7) (SEQ ID NO: 9)) decreased the hydrolysis rate significantly. Therefore, P6 (SEQ ID NO: 2) represents the optimum substrate for BoNT A protease activity.

In P5 (SEQ ID NO: 8), P6 (SEQ ID NO: 2), and P7 (SEQ ID NO: 9), E194 was replaced with alanine instead of glutamine. This change reduced synthesis cost, and as noted above, alanine and glutamine were equally effective at this location with regard to enhancing cleavage rate.

Table 2 lists various substituted peptides.

TABLE 2

| Sequence | | | | | |
|---|---|---|---|---|---|
| X1 X2 R I D X3 A N Q R A T X4 X5 | X1 | X2 | X3 | X4 | X5 |
| (SEQ ID NO: 1) | | | | | |
| KVRIDEANQRATKM | K | V | E | K | M |
| (SEQ ID NO: 10) | | | | | |
| KTRIDQANQRATKM | K | T | Q | K | M |
| (SEQ ID NO: 11) | | | | | |
| RTRIDQANQRATRM | R | T | Q | R | M |
| (SEQ ID NO: 12) | | | | | |
| RBRIDQANQRATRM | R | B | Q | R | M |
| (SEQ ID NO: 13) | | | | | |
| RVRIDQANQRATRM | R | V | Q | R | M |
| (SEQ ID NO: 14) | | | | | |
| RVRIDAANQRATRM | R | V | A | R | M |
| (SEQ ID NO: 2) | | | | | |

### Example 3

For assays utilizing HPLC analysis, reaction mixtures (30 μL) were incubated at 37° C. and contained 40 mM HEPES, 0.05% polysorbate-20, 1 mM dithiothreitol, 50 μM ZnCl₂, 0.5 mg/ml bovine serum albumin, and various concentrations of BoNT A Lc and substrate. Assays were stopped by acidification with trifluoroacetic acid and analyzed by HPLC (21, 24).

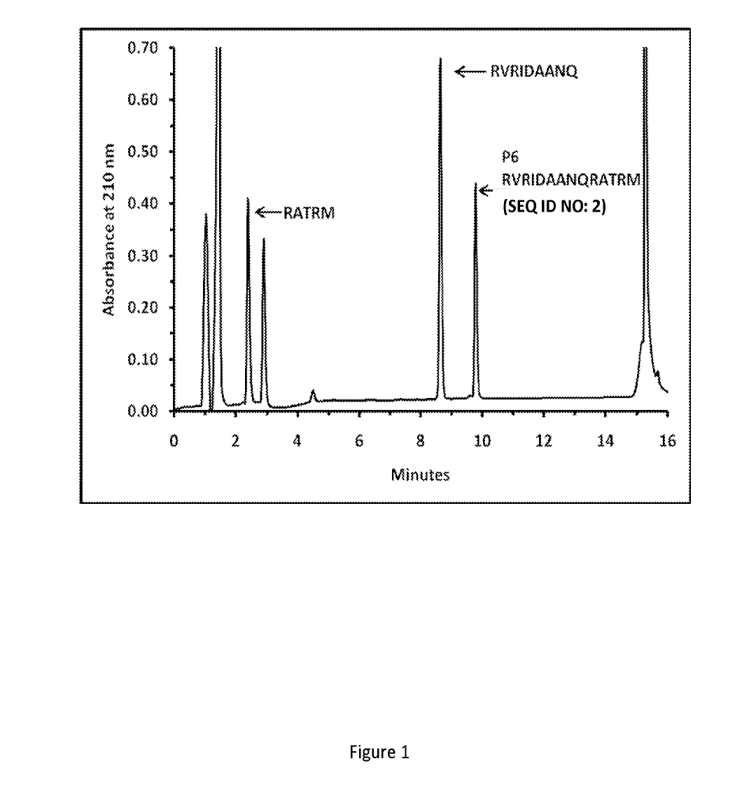

FIG. 1 depicts a HPLC-based assay of BoNT A light chain protease activity with P6 (SEQ ID NO: 2) as the substrate. P6 (SEQ ID NO: 2) (0.40 mM) was incubated with BoNT A light chain (BoNT A-Lc, the protease entity of botulinum neurotoxin) (20 nM) at 37° centigrade for 5 minutes at pH 7.3. The reaction was stopped by acidification and subjected to HPLC.

The column used for HPLC analysis was a Waters Associates Symmetry C18, 4.6×75 mm. The flow rate used was 1 ml/min and the temperature was 30 degrees C. Solvent A was 0.1% trifluoroacetic acid (TFA) in water, and solvent B was 70% acetonitrile/0.1% TFA. The column was equilibrated with 10% B. After sample injection, the column was maintained at 10% B for one minute, followed by a linear gradient to 36% B at 12.5 min. The column was then washed with 100% B for 6 min, followed by re-equilibration with 10% B. Various other HPLC protocols and columns capable of separating the various components of the sample may be used.

FIG. 1 is an HPLC chromatogram illustrating hydrolysis of P6 (SEQ ID NO: 2) catalyzed by BoNT A light chain. Hydrolysis products and the remaining substrate are labeled, and are well-resolved from each other. Unlabelled peaks are other assay components. The portions of P6 (SEQ ID NO: 2) that have been cleaved by BoNT A, RVRIDAANQ and RATRM, elute at a different time than full-length P6 (SEQ ID NO: 2). In this assay, 75% of the substrate P6 (SEQ ID NO: 2) was hydrolyzed by BoNT A. In contrast, under identical conditions, the widely-used BoNT A substrate peptide consisting of residues 187-203 of SNAP-25 ((P1) (SEQ ID NO: 4)) exhibited only 24% hydrolysis.

### Example 4

Kinetic constants were calculated from non-linear regression plots with the program Enzfitter (Biosoft, Cambridge, United Kingdom), including at least seven different substrate concentrations. In all cases, each reported value is the average of three independent experiments±standard deviation.

For determinations of kinetic constants, assays were incubated for various times, depending on substrate concentration, so that less than 10% of the substrate was hydrolyzed. Assays were stopped by acidification with trifluoroacetic acid and analyzed by HPLC (21, 24).

The superiority of P6 (SEQ ID NO: 2) as a substrate for BoNT A protease activity is further revealed by comparing P6 (SEQ ID NO: 2) kinetic constants with two other substrates which are commonly used in many laboratories to measure BoNT A protease activity. Kinetic constants of P6 (SEQ ID NO: 2) were determined and are compared with those of P1 (SEQ ID NO: 4) and full-length SNAP-25 in Table 3. For P6 (SEQ ID NO: 2), the Km of P6 (SEQ ID NO: 2) was 21-fold lower than the Km of the native-sequence P1 (SEQ ID NO: 4), and was statistically identical to that of full-length SNAP-25. SNAP-25 must be obtained by recombinant means and extensively purified. P6 (SEQ ID NO: 2) is ideal for use in BoNT A inhibitor discovery and other mechanistic studies. In contrast, the kcat of P6 (SEQ ID NO: 2) was 3.3 times that of P1 (SEQ ID NO: 4) and 33 times that of SNAP-25. Finally, the P6 (SEQ ID NO: 2) specificity constant (kcat/Km) was 71-fold higher than that of P1 (SEQ ID NO: 4) and 27-fold higher than that of SNAP-25. The kinetic constants of P6 (SEQ ID NO: 2) clearly demonstrate that P6 is the most efficient substrate described to date for the protease activity of BoNT A. P6 (SEQ ID NO: 2) contains only 14 residues and is easily synthesized in high yield. It is readily soluble in aqueous buffers up to at least 6 mM. P6 (SEQ ID NO: 2) is ideal for use in BoNT A inhibitor discovery and other mechanistic studies. The characteristics of P6 (SEQ ID NO: 2) afford greatly enhanced sensitivity and economy of use compared to other substrates for BoNT A protease activity.

TABLE 3
Kinetic constants of BoNT A substrates.

| | Kₘ | k_{cat} | k_{cat}/Kₘ |
|---|---|---|---|
| Substrate | (mM) | (sec⁻¹) | (M⁻¹sec⁻¹) |
| P6 | 0.052 ± 0.0044 | 76 ± 2.1 | 1.5 × 10⁶ |
| (SEQ ID NO: 2) | | | |
| P1 (SNAP-25 residues | 1.1 ± 0.10 | 23 ± 1.0 | 2.1 × 10⁴ |
| 187-203¹) (SEQ ID NO: 4) | | | |
| SNAP-25 residues 1-203² | 0.041 ± 0.010 | 2.3 ± 0.84 | 5.5 × 10⁴ |

¹From reference (25) Schmidt and Stafford, (2003) Appl. Env. Microbiol., 69(1): 297-303.
²From reference (26) Li et al. (2000) Biochemistry 39: 2399-2405.

### Example 5

P6 (SEQ ID NO: 2) is a superior substrate for BoNT A protease activity and flP6 ([DabcylK] (SEQ ID NO: 2) [SFC]) is an improved FRET substrate. A fluorophore (S-fluoresceinyl cysteine) and a quencher (N-[epsilon]-dabcyl-lysine) were added to the sequence of P6 (SEQ ID NO: 2) at the C-terminus and the N-terminus, respectively, to yield peptide flP6 ([DabcylK] (SEQ ID NO: 2) [SFC]). FlP6 ([DabcylK] (SEQ ID NO: 2) [SFC]) is a FRET substrate for the protease activity of BoNT A. In flP6 ([DabcylK] (SEQ ID NO: 2) [SFC]), the fluorophore is S-fluoresceinyl cysteine, abbreviated [SFC], and the quencher is N(epsilon)-(4-dimethylamino-azo-benzene-4′-carboxyl)-lysine, abbreviated [DabcylK]. The structure of flP6 ([DabcylK] (SEQ ID NO: 2) [SFC]) is: [DabcylK] R V R I D A A N Q R A T R M [SFC]. In an embodiment, the pair includes S-fluoresceinyl cysteine [SFC] as the fluorophore and N(epsilon)-(4-dimethylamino-azo-benzene-4′-carboxyl)-lysine [DabcylK] as the quencher. However, many other fluorophore-quencher pairs are known and are understood to be included here. Any BoNT A FRET substrate that includes the peptides disclosed below may be used.

(SEQ ID NO: 1)
X1 X2 R I D X3 A N Q R A T X4 X5

X1 through X5 are chosen from the following list:
X1 is K, R, ornithine, or 2,4-diaminobutanoic acid (dbu).
X2 is V, 2-aminobutanoic acid (B), I, L, or T.
X3 is Q, A, norvaline (nV), B, or E.
X4 is K, R, or dbu.
X5 is M or norleucine (nL).

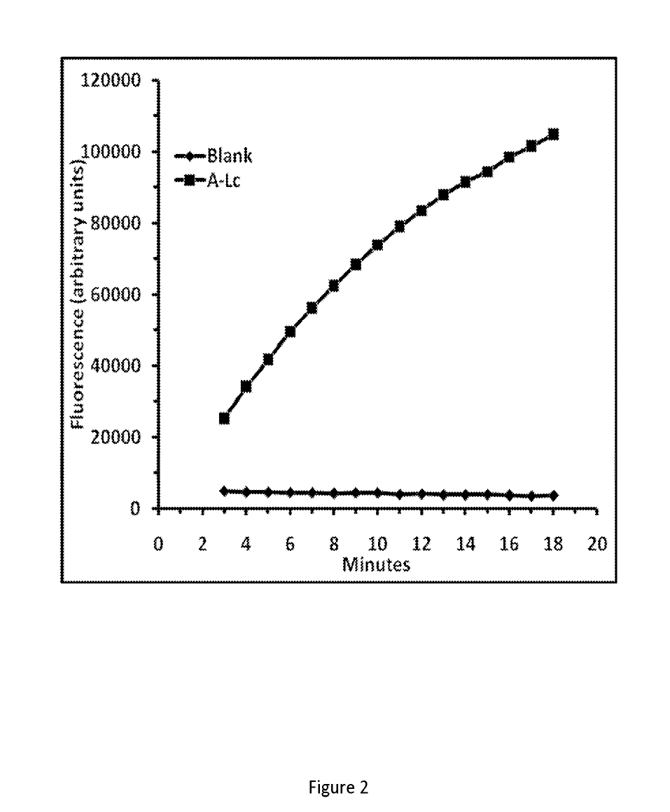

FlP6 ([DabcylK] (SEQ ID NO: 2) [SFC]) was used as a substrate to measure the protease activity of BoNT A-Lc. FIG. 2 depicts a plot that shows fluorescence values vs. time in the absence (Blank) and presence (A-Lc) of BoNT A light chain. Fluorescence was read at timed intervals on a Wallac 1420 Multiwell Counter equipped with an automated plate stacker (Perkin Elmer, Waltham, Mass.) and is expressed in arbitrary units. Excitation and emission wavelengths were 485 nm and 535 nm, respectively. The concentration of flP6 ([DabcylK] (SEQ ID NO: 2) [SFC]) was 10 μM and flP6 ([DabcylK] (SEQ ID NO: 2) [SFC]) was incubated with and without 5 nM BoNT A-Lc at pH 7.3 and 25° C. The assays were 40 μL and contained buffer components, BoNT A-Lc, and flP6 ([DabcylK] (SEQ ID NO: 2) [SFC]). The assay was done three times and the slope, representing the reaction rate, of the linear portion of each curve (3 min to 7 min) was determined. The average slope was 6381±1204 fluorescence units/min.

In FIG. 2, data points labeled “Blank” were obtained from incubation of 10 μM flP6 ([DabcylK] (SEQ ID NO: 2) [SFC]) without BoNT A-Lc. With BoNT A-Lc, fluorescence values did not change significantly during the assay, except for a very small amount of photobleaching, a phenomenon common to all fluorescent molecules. This shows that flP6 ([DabcylK] (SEQ ID NO: 2) [SFC]) is stable in the assay conditions, as there is no significant change in fluorescence. Data points labeled “A-Lc” were obtained from incubation of 10 μM flP6 ([DabcylK] (SEQ ID NO: 2) [SFC]) with 5 nM BoNT A-Lc. The increase in fluorescence shows that flP6 ([DabcylK] (SEQ ID NO: 2) [SFC]) is rapidly cleaved by BoNT A-Lc. In the presence of 5 nM light chain, a rapid increase in fluorescence was observed, reaching a value 28-fold higher than that of the blank at 15 minutes. Post-reaction analysis of the A-Lc assay mixture by HPLC revealed that 60% of the substrate flP6 ([DabcylK] (SEQ ID NO: 2) [SFC]) had been cleaved by BoNT A-Lc in 15 minutes.

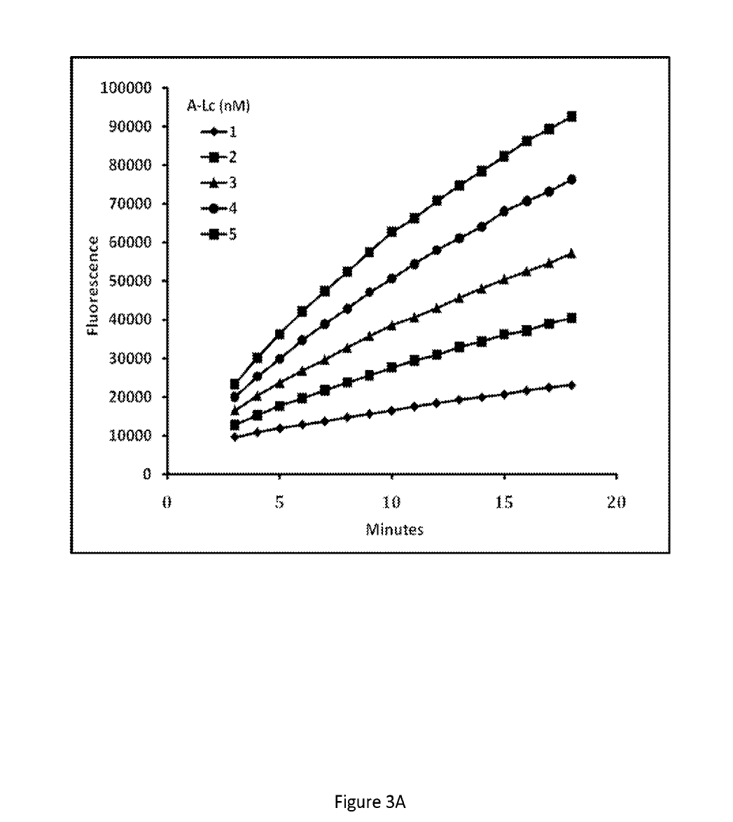

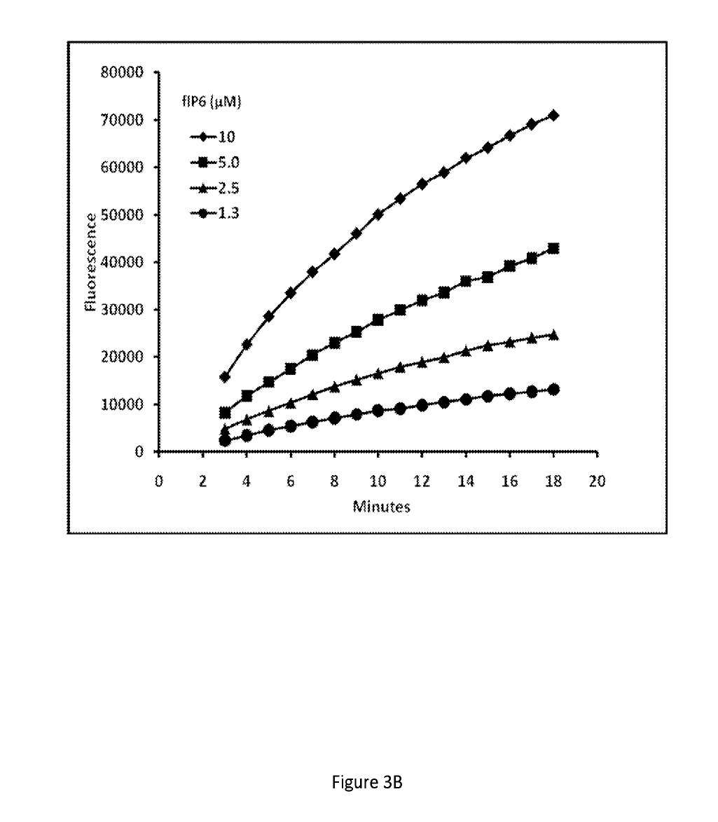

FIG. 3A depicts hydrolysis of 10 μM flP6 ([DabcylK] (SEQ ID NO: 2) [SFC]) catalyzed by various concentrations (1, 2, 3, 4, or 5 nM) of BoNT A light chain. Hydrolysis rates from the linear portions of the curves were 1030, 2220, 3265, 4712, and 6049 fluorescence units/min, for 1, 2, 3, 4, and 5 nM light chain, respectively. Although the lower limits of detection were not determined, it was clear from FIG. 3A that considerably lower light chain concentrations could be employed with extended reaction times.

FIG. 3B depicts hydrolysis of various flP6 ([DabcylK] (SEQ ID NO: 2) [SFC]) concentrations (1.3, 2.5, 5.0, or 10 μM), catalyzed by 4 nM BoNT A light chain. Hydrolysis rates were 980, 1800, 2999, and 5537 fluorescence units/min for 1.3, 2.5, 5.0, and 10 μM flP6 ([DabcylK] (SEQ ID NO: 2) [SFC]), respectively. These findings show that practical assays are possible even with very low concentrations of flP6 ([DabcylK] (SEQ ID NO: 2) [SFC]), an important consideration with respect to suitability for use in high-throughput assays.

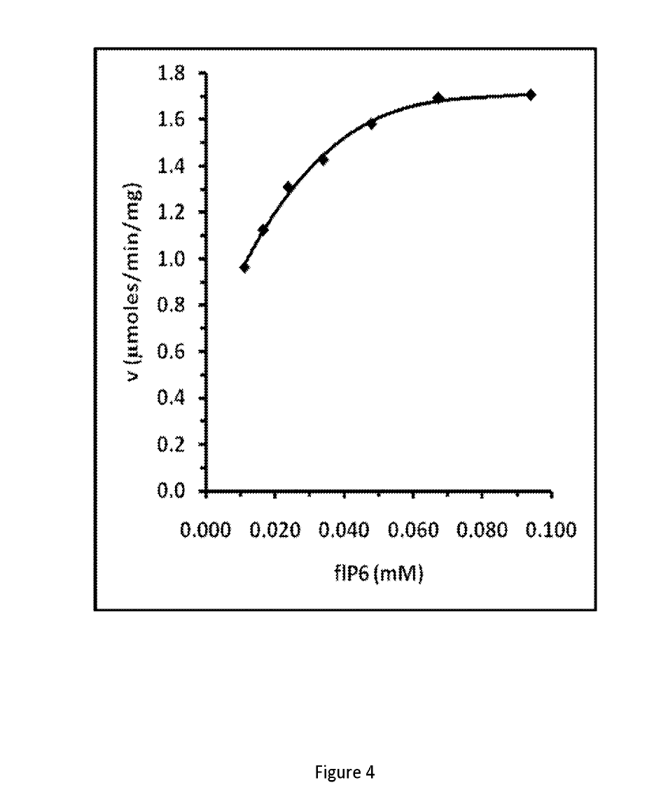

The substrate flP6 ([DabcylK] (SEQ ID NO: 2) [SFC]) exhibited saturable Michaelis-Menten kinetics, shown in FIG. 4. FIG. 4 depicts initial hydrolysis rates (v) at various flP6 ([DabcylK] (SEQ ID NO: 2) [SFC]) concentrations, catalyzed by 4 nM BoNT A light chain. To avoid artifacts due to the inner filter effect, these assays were done by HPLC.

The kinetic constants of flP6 ([DabcylK] (SEQ ID NO: 2) [SFC]), determined in triplicate, are shown in Table 4, and compared with two other BoNT A FRET substrates. In Fl-A (SEQ ID NO: 15), a BoNT A FRET substrate (25), the fluorophore was methyl-dimethylaminocoumarin and the quencher was a dinitrophenyl group. Fl-A (SEQ ID NO: 15) displayed typical Michaelis-Menten kinetics, and using the assay conditions described herein (10 μM substrate, 5 nM BoNT A light chain), was cleaved at a rate similar to flP6 ([DabcylK] (SEQ ID NO: 2) [SFC]). However, because the fluorescence yield of fluorescein is greater than that of methyl-dimethylaminocoumarin, the net fluorescence increase of flP6 ([DabcylK] (SEQ ID NO: 2) [SFC]) observed in the assay was seven-fold higher than Fl-A (SEQ ID NO: 15). Therefore, flP6 ([DabcylK] (SEQ ID NO: 2) [SFC]) would be preferred when high sensitivity is needed. In addition, the higher excitation wavelength of flP6 ([DabcylK] (SEQ ID NO: 2) [SFC]) (485 nm), than Fl-A (SEQ ID NO: 15)) (398 nm) will diminish the number of spurious results when screening libraries of organic compounds, which may be fluorescent themselves and/or quench substrate fluorescence. dnpK is Nε-(2,4-dinitrophenyl)lysine. DaciaC is the product of the reaction of the cysteine sulfhydryl group with 3-iodoacetamido-4-methyl-7-dimethylamine coumarin.

TABLE 4
Kinetic constants of BoNT A FRET substrates.

| | Kₘ | k_{cat} | k_{cat}/Kₘ |
|---|---|---|---|
| Substrate | (mM) | (sec⁻¹) | (M⁻¹sec⁻¹) |
| flP6¹ | 0.013 ± 0.0015 | 1.7 ± 0.090 | 1.3 × 10⁵ |
| Fl-A² | 0.096 ± 0.010 | 7.2 ± 0.40 | 7.5 × 10⁴ |
| Boldt et al.³ | ≧0.1 | ND | 4.3 × 10³ |

¹([DabcylK] (SEQ ID NO: 2) [SFC])
²From reference (25) Schmidt and Stafford, (2003), Appl. Env. Microbiol. 69(1): 297-303. Fl-A is SNRTRIDEAN[dnpK]RA[daciaC]RML. (SEQ ID NO: 15)
³From reference (27) Boldt et al., (2006), J. Comb. Chem., 8(4): 513-21. The sequence in Boldt et al. is FITC-T(D)RIDQANQ(Ψ)RATK(εDabcyl)nL (synthesized by the authors and referred to as SNAPtide ®). (SEQ ID NO: 16)

Table 4 depicts that FRET substrates based on the peptides disclosed herein offer the same advantages as those described above for P6 (SEQ ID NO: 2), compared to other FRET substrates for BoNT A protease activity. For example, in contrast to flP6 ([DabcylK] (SEQ ID NO: 2) [SFC]), another FRET substrate for BoNT A, the substrate described by Boldt et al. (27) (SEQ ID NO: 16), was reported to exhibit non-saturable kinetics, inasmuch as the rate of hydrolysis continued to increase linearly up to the solubility limit of the substrate. A Km of at least 100 μM was estimated, which is 8-fold higher than that of flP6 ([DabcylK] (SEQ ID NO: 2) [SFC]) and well above the useful concentration range for FRET substrates (usually up to 20 μM). The Km of flP6 ([DabcylK] (SEQ ID NO: 2) [SFC]), 13 μM, is well within that range. In the publication (27), a value for kcat was not reported, but kcat/Km was 4300, 30-fold lower than that of flP6 ([DabcylK] (SEQ ID NO: 2) [SFC]). The kinetic properties of flP6 ([DabcylK] (SEQ ID NO: 2) [SFC]) are superior to those of the FRET substrate Boldt et al. (27) (SEQ ID NO: 16). FlP6 ([DabcylK] (SEQ ID NO: 2) [SFC]) is an exceptional FRET substrate for BoNT A inhibitor discovery and other BoNT A enzymatic studies.

### Example 6

The substrates will be modified to include a fluorescent group on one side of the cleavage site and a cysteine or biotin on the other side. The substrates will be bound to multiwell plates or other surfaces (such as beads or resins) that will be activated with N-ethylmaleimide or streptavidin, respectively. The protease activity of BoNT A will cleave the immobilized substrate, releasing fluorescence into solution, which will be monitored with suitable instrumentation. In some assays, the increase in fluorescence with time can be monitored in situ. In others, the solution will be separated from the solid phase and the fluorescence of the solution measured. Fluorescence of the solution will increase as the amount of substrate cleaved increases. Other means of coupling proteins or peptides will also be used. For example, instead of a cysteine and n-ethylmaleimide or biotin and streptavidin, one of the following means of coupling can be used, cysteine and iodoacetate or iodoacetamide, glutathione-S-transferase and glutathione, a His tag and a metal chelate bound to the surface.

### Example 7

Structural modifications will be made to the substrate(s) described herein in order to enable intracellular localization of the substrate. Intracellular localization of the substrates will allow assay of botulinum protease activity inside cells. Examples of such modifications include, but are not limited to, addition of the HIV TAT transmembrane sequence to a substrate, or addition of the peptide called “penetratin” to a substrate. Any method of allowing the substrate be located inside the cell wall may be used.

Intracellular localization of the substrate will also allow the substrates to be used as competitive inhibitors to compete with endogenous SNAP-25 for the BoNT-A Lc active site. The binding of substrates to BoNT-A Lc will protect endogenous SNAP-25 from cleavage to an extent. These competitive inhibitors may be given to a subject in need of treatment of a condition due to botulinum toxin. In an embodiment, multiple doses of the substrates may be administered to the subject. In an embodiment, the dose or doses may be administered enterally, parenterally, or other suitable method. The dose or doses may be in the form of a liquid, tablet, capsules, or other suitable form. The dosage given is that necessary to provide a partial or full inhibition of BoNT A.

### Example 8

A kit will used to detect the presence of serotype A botulinum neurotoxin in human or animal samples (including, but not limited to, blood, stool, organs and tissues), in environmental samples (including, but not limited to, soil, food, and water), and in other samples. In addition to detection, the kit may also be used to quantify the amount of serotype A botulinum neurotoxin in the sample.

In one embodiment, the kit will comprise the following:
(a) A vial containing a pH-buffering compound in dry form. The composition of this buffer is such that, upon dissolving in water, the pH is 7.5±0.5. Suitable examples include sodium hydroxyethylpiperazine sulfonate, commonly abbreviated as “HEPES”, and sodium phosphate. The vial will also contain one or more agent(s) known to stabilize BoNT A, such as bovine serum albumin. Before use, the buffer is dissolved in a suitable volume of purified water (g). Polysorbate 20 (i) is added to a final concentration of 0.05-0.10% (v/v).
(b) A vial containing a pH-buffering compound in dry form. The composition of this buffer is such that, upon dissolving in water, the pH is 7.5±0.5. Suitable examples include sodium hydroxyethylpiperazine sulfonate, commonly abbreviated as “HEPES”, and sodium phosphate. The vial will also contain, in dry form, a reducing agent (for example, dithiothreitol or tris-(carboxyethyl)-phosphine), and a zinc salt (for example, zinc chloride or zinc acetate). The vial will also contain one or more agent(s) known to stabilize BoNT A, such as bovine serum albumin. Before use, the buffer is dissolved in a suitable volume of purified water (g). Polysorbate 20 (i) is added to a final concentration of 0.05-0.10% (v/v).
(c) A vial containing one of the FRET substrates described herein, in dry form. Before use, the substrate is dissolved in an appropriate volume of dimethylsulfoxide (h).
(d) Polymeric beads (or other suitable solid material), coated with antibodies specific for BoNT A.
(e) The same solid material as in (d), coated with the same type of immunoglobulins as in (d), but with NO antibodies to BoNT A.
(f) A vial containing a known quantity of lyophilized BoNT A, with suitable stabilizing excipients (if needed), for preparation of standard curves. Before use, lyophilized BoNT A is dissolved in buffer (a).
(g) A bottle containing highly purified water, free of contaminants that could compromise the assay.
(h) A bottle containing highly purified dimethylsulfoxide, free of contaminants that could compromise the assay.
(i) A bottle containing a suitable non-ionic detergent (for example, Polysorbate 20), free of contaminants that could compromise the assay.

In one embodiment, the kit would be used as follows:
(1) The sample in question is dissolved in or extracted with buffer (a), then mixed with the antibody-coated beads (d). Any BoNT A in the sample will be captured and immobilized on the beads by the antibodies. This is called the test assay.
(2) After an appropriate incubation time, the beads are then washed with buffer (a) to remove unwanted material in the sample.
(3) Simultaneously, a second assay is prepared in the same way, but using beads (e). This is called the control assay.
(4) After washing, the beads from the test assay and from the control assay are suspended, in separate tubes, in a solution containing buffer (b) and the FRET substrate (c).
(5) After a suitable incubation period, the solutions are separated from the beads and the fluorescence intensities of the solutions are measured. If the fluorescence of the assay that contained beads (d) (the test assay) is higher than the assay that contained beads (e) (the control assay), the presence of BoNT A in the test sample is indicated.
(6) The concentration of BoNT A in the test sample may be determined by comparison of the fluorescence intensity of the test assay with a standard curve using known concentrations of BoNT A, prepared using vial (f)

In sum, the HPLC-based and fluorigenic BoNT A substrates reported here represent significant improvements, compared to current widely-used BoNT A substrates, in terms of hydrolysis rates, binding constants, assay sensitivity, and ease of synthesis. These improvements and characteristics result from non-conservative amino acid substitutions and truncations to a peptide which, in its original form, corresponded to residues 187-203 of the neuronal protein, SNAP-25. With regard to FRET substrates, these advantages are particularly apparent when comparing the kinetic properties of flP6 ([DabcylK] (SEQ ID NO: 2) [SFC]) with those of a previously-described BoNT A FRET substrate from another laboratory (27). The substrates disclosed herein are useful in BoNT A mechanistic studies, high-throughput inhibitor searches, and in therapy for botulism. The substrates could also be employed to further enhance the sensitivity of a recently-reported attomolar-level BoNT A detection system (29).

### REFERENCES

1. Arnon, S. S., et al., 2001. Botulinum toxin as a biological weapon. J. Am. Med. Assoc. 285:1059-1070.
2. Schiavo, G., et al, Physiol. Rev. 80:717-766.
3. Simpson, L. L. 2004. Identification of the major steps in botulinum toxin action, Ann. Rev. Pharmacol. Toxicol. 44:167-193.
4. Ahmed, S. A. and L. A. Smith. 2000. Light chain of botulinum A neurotoxin expressed as an inclusion body from a synthetic gene is catalytically and functionally active. J. Prot. Chem. 19:475-487.
5. Gilsdorf, J., N. Gul, and L. A. Smith. 2006. Expression, purification, and characterization of Clostridium botulinum type B light chain. Protein Expr. Purif. 46:256-267.
6. Cherington, M. 2004. Botulism: update and review. Semin. Neurol. 24:155-163.
7. Foster, K. A., P. Hambleton, and C. C. Shone (ed.). 2007. Treatments From Toxins. CRC Press, New York, NY.
8. Turton, K., J. A. Chaddock, and K. R. Acharya. 2002. Botulinum and tetanus neurotoxins: structure, function, and therapeutic utility. Trends Biochem. Sci. 27:552-558.
9. Ansiaux, R., C. et al., 2006. Botulinum toxin potentiates cancer radiotherapy and chemotherapy. Clin. Cancer Res. 12:1276-1283.
10. Bade, S., et al., 2004. Botulinum neurotoxin type D enables cytosolic delivery of enzymatically active cargo proteins to neurons via unfolded translocation intermediates. J. Neurochem. 91:1461-1472.
11. Foran, P. 2007. Use of neurotoxins as drug delivery systems, p. 213-253. In K. A. Foster, P. Hambleton, and C. C. Shone (ed.), Treatments From Toxins. CRC Press, New York, N.Y.
12. Goodnough, M. C., et al., 2002. Development of a delivery vehicle for intracellular transport of botulinum neurotoxin antagonists. FEBS Lett. 513:163-168.
13. Zdanovskaia, M. V., et al, 2000. Recombinant derivatives of clostridial neurotoxins as delivery vehicles for proteins and small organic molecules. J. Protein Chem. 19:699-707.
14. Wein, L. M., and Y. Liu. 2005. Analyzing a bioterror attack on the food supply: the case of botulinum toxin in milk. Proc. Natl. Acad. Sci. U. S. A. 102:9984-9989.
15. Keller, J. E., et al., 2004. Uptake of botulinum neurotoxin into cultured neurons. Biochemistry. 43:526-532.
16. Burnett, J. C., et al., 2003. Novel small molecule inhibitors of botulinum neurotoxin protease activity. Biochem. Biophys. Res. Comm. 310:85-94.
17. Burnett, J. C., et al., 2007 Inhibition of Metalloprotease botulinum serotype A from a pseudo-peptide binding mode to a small molecule that is active in primary neurons. J. Biol. Chem. 282:5004-5014.
18. Burnett, J. C., et al., 2005. Conformational sampling of the botulinum neurotoxin serotype A light chain: implications for inhibitor binding. Bioorg. Med. Chem. 13:333-341.
19. Merino, I., et al., 2006. Bis-imidazoles as molecular probes for peripheral sites of the zinc endopeptidase of botulinum neurotoxin serotype A. Bioorg. Med. Chem. 14:3583-3591.
20. Park, J. G., et al., 2006. Serotype-selective, small-molecule inhibitors of the zinc endopeptidase of botulinum neurotoxin serotype A. Bioorg. Med. Chem. 14:395-408.
21. Schmidt, J. J. and K. A. Foster. 2007. Inhibitors of clostridial neurotoxin protease activities, p. 107-138. In K. A. Foster, P. Hambleton, and C. C. Shone (ed.), Treatments From Toxins. CRC Press, New York, N.Y.
22. Schmidt, J. J. and R. G. Stafford. 2005. Botulinum neurotoxin serotype F: identification of substrate recognition requirements and development of inhibitors with low nanomolar affinity. Biochemistry 44: 4067-4073.
23. Schmidt, J. J. and R. G. Stafford. 2002. A high-affinity competitive inhibitor of type A botulinum neurotoxin metalloprotease activity. FEBS Lett. 532:423-426.
24. Schmidt, J. J. and K. A. Bostian. 1997. Endoproteinase activity of type A botulinum neurotoxin: substrate requirements and activation by serum albumin. J. Prot. Chem. 16:19-26.
25. Schmidt, J. J., and R. G. Stafford. 2003. Fluorigenic substrates for the protease activities of botulinum neurotoxins, serotypes A, B, and F. Appl. Environ. Microbiol. 69:297-303.
26. Li, L., et al., 2000. Probing the mechanistic role of glutamate residue in the zinc-binding motif of type A botulinum neurotoxin light chain. Biochemistry 39:2399-2405.
27. Boldt, G. E., et al., 2006. Synthesis, characterization, and development of a high-throughput methodology for the discovery of botulinum neurotoxin A inhibitors. J. Comb. Chem. 8:513-521.
28. Breidenbach, M. A., and A. T. Brunger. 2004. Substrate recognition strategy for botulinum neurotoxin serotype A. Nature 432:925-929.
29. Bagramyan, K., et al., 2008. Attomolar Detection of Botulinum Toxin Type A in Complex Biological Matrices. PLoS ONE 3(4): e2041. doi:10.1371/journal.pone.0002041.
30. Kistner, A. and Habermann, E. 1992. Naunyn Schmiedebergs Arch Pharmacol. 338:99-106.
31. Barr, J. R., et al., 2005. Botulinum Neurotoxin Detection and Differentiation by Mass Spectrometry. Emerging Infectious Diseases 11(10): 1578-1583.
32. Dong, M. et al., 2006. Science. 312(5773):593-596.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. An isolated peptide comprising the amino acid sequence of SEQ ID NO: 1,
(SEQ ID NO: 1)
X1 X2 R I D X3 A N Q R A T X4 X5
wherein each of X1 through X5 is chosen from the following:
X1 is K, R, ornithine, or 2, 4-diaminobutanoic acid (dbu);
X2 is V, 2-aminobutanoic acid (B), I, L, or T;
X3 is Q, A, norvaline (nV), B, or E;
X4 is K, R, or dbu; and
X5 is M or norleucine (nL);
except wherein X1=K, X2=T, X3=E, X4=K, and X5=M;
X 1=K, X2=T, X3=Q, X4=K, and X5=M; or
X1=R, X2=T, X3=Q, X4=R, and X5=M.

2. The isolated peptide of claim 1, wherein the isolated peptide is modified with a fluorophore located on one side of a BoNT A cleavage site and a quencher located on the other side of the BoNT A cleavage site.

3. The isolated peptide of claim 1, comprising the amino acid sequence
(SEQ ID NO: 2)
R V R I D A A N Q R A T R M

4. The isolated peptide of claim 3, wherein the isolated peptide is modified with a fluorophore located on one side of a BoNT A cleavage site and a quencher located on the other side of the BoNT A cleavage site.

5. The isolated peptide of claim 4, wherein the fluorophore is located at one terminus and a quencher is located at the other terminus.

6. The isolated peptide of claim 5, wherein DabcylK is located at the N-terminus and S-fluoroceinyl cysteine is located at the C-terminus.

7. The isolated peptide of claim 1, comprising the amino acid sequence
(SEQ ID NO: 3)
R V R I D A A N Q R A T R nL

8. The isolated peptide of claim 7, wherein the isolated peptide is modified with a fluorophore located on one side of a BoNT A cleavage site and a quencher located on the other side of the BoNT A cleavage site.

9. The isolated peptide of claim 8, wherein the fluorophore is located at one terminus and a quencher is located at the other terminus.

10. The isolated peptide of claim 9, wherein DabcylK is located at the N-terminus and S-fluoroceinyl cysteine is located at the C-terminus.

11. A kit to detect the presence of BoNT A in a sample comprising
the isolated peptide of claim 2;
polymeric beads coated with antibodies specific for BoNT A; and
polymeric beads coated with immunoglobulins not specific for BoNT A.

12. The kit of claim 11, further comprising lyophilized BoNT A Lc.

13. The kit of claim 11, further comprising a pH-buffering compound in a first container.

14. The kit of claim 13, wherein the pH-buffering compound is selected from the group consisting of sodium hydroxyethylpiperazine sulfonate (HEPES) and sodium phosphate.

15. The kit of claim 13, wherein the pH-buffering compound is in dry form.

16. The kit of claim 11, further comprising bovine serum albumin.

17. The kit of claim 11, further comprising polysorbate 20.

18. The kit of claim 17, wherein the polysorbate 20 is added to a final concentration of 0.05-0.10% (v/v).

19. The kit of claim 11, further comprising a reducing agent in a first container.

20. The kit of claim 19, wherein the reducing agent is selected from the group consisting of dithiothreitol and tris-(carboxyethyl)-phosphine.

21. The kit of claim 11, further comprising a zinc salt in a first container.

22. The kit of claim 21, wherein the zinc salt is selected from the group consisting of zinc chloride and zinc acetate.

23. The kit of claim 11, further comprising purified water in a second container.

24. The kit of claim 11, further comprising purified dimethylsulfoxide in a third container.

25. The kit of claim 11, wherein the lyophilized BoNT A Lc comprises a stabilizing excipient.

26. The kit of claim 11, wherein the isolated peptide comprises the amino acid sequence of SEQ ID NO: 1,
(SEQ ID NO: 1)
X1 X2 R I D X3 A N Q R A T X4 X5
wherein each of X1 through X5 is chosen from the following:
X1 is K, R, ornithine, or 2, 4-diaminobutanoic acid (dbu);
X2 is V, 2-aminobutanoic acid (B), I, L, or T;
X3 is Q, A, norvaline (nV), B, or E;
X4 is K, R, or dbu; and
X5 is M or norleucine (nL);
except wherein X1=K, X2=T, X3=E, X4=K, and X5=M;
X1=K, X2=T, X3=Q, X4=K, and X5=M; or
X1=R, X2=T, X3=Q, X4=R, and X5=M is in dry form.

27. A method of detecting the presence of BoNT A in a sample comprising
placing the sample in solution in a pH-buffering compound;
mixing the sample in the pH-buffering compound with polymeric beads coated with antibodies specific for BoNT A to provide a test assay;
mixing the sample in the pH-buffering compound with polymeric beads with immunoglobulins not specific for BoNT A to provide a control assay;
incubating the sample with the pH-buffering compound with polymeric beads coated with antibodies specific for BoNT A to provide a test assay;
incubating the sample with the pH-buffering compound with polymeric beads with immunoglobulins not specific for BoNT A to provide a control assay;
washing the polymeric beads coated with antibodies specific for BoNT A with the pH-buffering compound;
washing the polymeric beads without antibodies with the pH-buffering compound;
suspending the beads from the test assay in a first container comprising the pH-buffering compound, dithiothreitol, and a zinc salt;
suspending the beads from the control assay a second container comprising the pH-buffering compound, dithiothreitol, and a zinc salt;
adding the isolated peptide of claim 2 to the test assay and control assay;
incubating the isolated peptide of claim 2 with the beads from the test assay;
incubating the isolated peptide of claim 2 with the beads from the control assay;
separating the beads from the test assay from a test assay solution comprising the isolated peptide of claim 2;
separating the beads from the control assay from a control assay solution comprising the isolated peptide of claim 2;
measuring fluorescence intensity of the test assay solution;
measuring fluorescence intensity of the control assay solution;
comparing the fluorescence of the test assay solution and the control assay solution; and
determining whether BoNT A is present in the sample by whether the fluorescence of the test assay solution is higher than the fluorescence of the control assay solution.

28. The method of claim 27, further comprising adding bovine serum albumin to the pH buffering compound.

29. The method of claim 27, further comprising adding polysorbate 20 present to the pH buffering compound.

30. The method of claim 29, wherein the polysorbate 20 is added to a final concentration of 0.05-0.10%.

31. A method of determining the concentration of BoNT A in a test sample comprising
placing the sample in solution in a pH-buffering compound;
mixing the sample in the pH-buffering compound with polymeric beads coated with antibodies specific for BoNT A to provide a test assay;
mixing the sample in the pH-buffering compound with polymeric beads with immunoglobulins not specific for BoNT A to provide a control assay;
incubating the sample with the pH-buffering compound with polymeric beads coated with antibodies specific for BoNT A to provide a test assay;
incubating the sample with the pH-buffering compound with polymeric beads with immunoglobulins not specific for BoNT A to provide a control assay;
washing the polymeric beads coated with antibodies specific for BoNT A with the pH-buffering compound;
washing the polymeric beads with immunoglobulins not specific for BoNT A with the pH-buffering compound;
suspending the beads from the test assay in a first container comprising the pH-buffering compound, dithiothreitol, and a zinc salt;
suspending the beads from the control assay a second container comprising the pH-buffering compound, dithiothreitol, and a zinc salt;
adding the isolated peptide of claim 2 to the test assay and control assay;
incubating the isolated peptide of claim 2 with the beads from the test assay;
incubating the isolated peptide of claim 2 with the beads from the control assay;
separating the beads from the test assay from a test assay solution comprising the isolated peptide of claim 2;
separating the beads from the control assay from a control assay solution comprising the isolated peptide of claim 2;
measuring fluorescence intensity of the test assay solution;
measuring fluorescence intensity of the control assay solution;
comparing the fluorescence of the test assay solution and the control assay solution; and
determining the concentration of BoNT A in the sample by comparison of fluorescence intensity of the test assay solution with a standard curve prepared using known concentrations of BoNT A Lc.

32. The method of claim 31, further comprising adding bovine serum albumin to the pH buffering compound.

33. The method of claim 31, further comprising adding polysorbate 20 present to the pH buffering compound.

34. The method of claim 33, wherein the polysorbate 20 is added to a final concentration of 0.05-0.10%.

35. A method for measuring the activity of BoNT A comprising
incubating the isolated peptide of claim 1 with BoNT A to form a sample;
injecting the sample onto an HPLC column;
preparing a chromatogram of the elution of various components of the sample;
analyzing the chromatogram to determine how much of the isolated peptide of claim 1 was cleaved based upon the size of peaks correlating to the isolated peptide of claim 1 and cleaved portions of the isolated peptide of claim

36. A method for identifying a BoNT A inhibitor comprising
incubating BoNT A with a potential inhibitor to form a first sample;
incubating BoNT A without a potential inhibitor to form a second sample;
adding the isolated peptide of claim 1 to the first sample;
adding the isolated peptide of claim 1 to the second sample;
stopping the reactions by adding acid to the first and second sample;
injecting the first sample onto an HPLC column;
preparing a chromatogram of the elution of various components of the first sample;
injecting the second sample onto an HPLC column;
preparing a chromatogram of the elution of various components of the second sample;
analyzing the chromatogram for the first sample and the second sample to determine how much of the isolated peptide of claim 1 was cleaved based upon the size of peaks correlating to the isolated peptide of claim 1 and cleaved portions of the isolated peptide of claim 1; and
determining that a potential inhibitor of BoNT A is a BoNT A inhibitor if the size of the peak correlating to the isolated peptide of claim 1 for the first sample is taller than the size of the peak for the isolated peptide of claim 1 the second sample.

37. A method of treating an individual in need of treatment for a disorder due to BoNT A comprising administering a composition comprising the isolated peptide of claim 1.
